(19) United States
(12) Patent Application Publication
(10) Pub. No.: US 2013/0274847 A1
(43) Pub. Date: Oct. 17, 2013

(54) MRI Compatible Leadless Cardiac Pacemaker

(71) Applicant: Alan Ostroff, Pleasanton, CA (US)

(72) Inventor: Alan Ostroff, Pleasanton, CA (US)

(21) Appl. No.: 13/915,560

(22) Filed: Jun. 11, 2013

Related U.S. Application Data
(62) Division of application No. 12/568,513, filed on Sep. 28, 2009, now abandoned.

(51) Int. Cl. A61N 1/05

(52) U.S. Cl. CPC A61N 1/059
USPC 607/127; 607/122; 607/129; 607/131

(57) ABSTRACT

An implantable battery powered leadless pacemaker or biostimulator is provided that may include any of a number of features. One feature of the biostimulator is that it safely operates under a wide range of MRI conditions. One feature of the biostimulator is that it has a total volume small enough to avoid excessive image artifacts during a MRI procedure. Another feature of the biostimulator is that it has reduced path lengths between electrodes to minimize tissue heating at the site of the biostimulator. Yet another feature of the biostimulator is that a current loop area within the biostimulator is small enough to reduce an induced current and voltage in the biostimulator during MRI procedures. Methods associated with use of the biostimulator are also covered.

### CROSS REFERENCE TO RELATED APPLICATIONS

This application is a divisional of U.S. application Ser. No. 12/568,513 filed on Sep. 28, 2009, which application is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to leadless cardiac pacemakers, and more particularly, to operating leadless cardiac pacemakers safely in a patient over a wide range of MRI conditions.

### BACKGROUND OF THE INVENTION

Magnetic Resonance Imaging (MRI) has become an important diagnostic tool used by physicians. However, the use of MRI is contraindicated by pacemaker manufacturers since MRI can be unsafe for patients with implanted pacemakers.

MRI generates cross-sectional images of the human body by first aligning hydrogen nuclei (protons) in one of two possible orientations using a strong, uniform, static magnetic field. Next a radio frequency (RF) signal at the appropriate resonant frequency is applied, which forces a spin transition of the hydrogen protons between the possible orientations. The spin transitions create a signal that can be detected by a receiving coil and processed to create the MRI image. MRI equipment generates three types of fields that can affect implantable pacemakers, including (1) a Static Magnetic Field, (2) a Pulsed Gradient Field, and (3) a RF Field.

The static magnetic field typically ranges from 0.2 to 3.0 T, but will probably exceed this value in subsequent MRI equipment generations. The static magnetic field can result in a magnetic force and torque component with implantable pacemakers due to the presence of ferromagnetic materials used in the construction of the implant. Additionally, many conventional implantable pacemakers contain a static magnetic field sensor, typically a reed switch, MEMS sensor, or giant magnetoresistance sensor, which is typically used to inactivate the sensing function of a pacemaker. The static magnetic field is typically more than sufficient to activate the implantable pacemaker's magnetic sensor, causing the pacemaker to revert to asynchronous pacing. This switch from normal inhibited mode pacing to asynchronous mode pacing can result in tachycardia leading to ventricular fibrillation should the pacemaker fire into the “vulnerable phase” of the cardiac cycle.

The pulsed gradient field is typically characterized by a magnetic field strength gradient of up to 50 mT/m, a slew-rate of up to 20 T/sec (limit set to avoid peripheral nerve stimulation) and a frequency in the kilohertz range. The effects of the pulsed gradient field in an implanted pacemaker are induced currents in the loop area defined by the pacemaker lead and return path from the distal pacing electrode back to the implanted subcutaneous pulse generator. Induced currents and voltages in a pacemaker can cause inappropriate sensing and triggering and even stimulation. The loop area for a typical left-sided pacemaker implant was found by the AAMI EMC Task Force to be typically on the order of 200 cm² with the worst-case loop area being twice that value. For a conventional pacemaker, the induced voltage can be as large as 320 mV peak or 640 mV peak-to-peak.

The RF field can result in tissue heating at the site of the electrode tip of an implanted pacemaker. RF energy, up to 35 kW peak and 1 kW average can be radiated to the body at a frequency known as the Larmor frequency, which corresponds to the resonant frequency for the absorption of energy by the protons for a particular nucleus. The Larmor frequency is approximately 64 MHz for field strength of 1.5 T. In vivo measurements in a pig model have been shown to increase the temperature by as much as 20° C. near the pacing tip of an implanted pacemaker during exposure to 1.5 T MRI.

A pacemaker in the MRI field can also distort the field creating image artifacts. These artifacts have been measured with conventional pacemakers and lead systems to be as large as 177 cm² due mostly to the subcutaneously implanted pulse generator. The primary factors that affect the artifact size include the magnetic susceptibility and the mass of the materials used in the pulse generator.

Some of the current solutions to these problems are using RF filtering and shielding within the pacemaker to attenuate the induced currents and voltages in the pacing leads due to the pulsed RF magnetic fields, using a fiber optic cable to eliminate the induced currents from the pulsed RF magnetic field, using an isolation system in conjunction with magnetic and RF sensors dynamically to attenuate or eliminate induction loops, and using a band-stop filter to block EMI. Some of these provide for safe operation under MRI conditions, but only over a limited range of MRI conditions.

Accordingly, the present invention is directed to provide an implantable cardiac pacemaker system for safe operation during MRI imaging over a wide range of MRI conditions.

### SUMMARY OF THE INVENTION

The present invention relates to leadless cardiac pacemakers, and more particularly, to operating leadless cardiac pacemakers safely in a patient over a wide range of MRI conditions.

One aspect of the invention provides a leadless biostimulator, comprising a housing adapted to be implanted in or on a human heart, the housing having a total volume less than 1.5 cm³, a first electrode and a second electrode coupled to the housing, a pulse generator disposed in the housing and electrically coupled to the first and second electrodes, the pulse generator configured to generate and deliver electrical pulses to heart tissue via the first and second electrodes, and a battery disposed in the housing and coupled to the pulse generator, the battery configured to supply energy for electrical pulse generation.

In some embodiments, the leadless biostimulator the total volume of the housing can be less than 1.1 cm³.

In other embodiments, the first electrode is spaced less than 2 cm from the second electrode. The first or second electrode can comprise a pace/sense electrode. In some embodiments, the second electrode can comprise a return electrode. The second electrode can also comprise a can electrode. In some embodiments one or both of the electrodes can comprise a low-polarization coating.

The first electrode can be disposed on a flexible member. In some embodiments, the flexible member can comprise a fixation helix. In other embodiments, the fixation helix can be at least partially coated with an insulator, wherein the first electrode can comprise an uncoated portion of the fixation helix.

Another aspect of the invention provides an insulator disposed between the first and second electrodes. The insulator can be a coated portion of the housing. In some embodiments, the first electrode can be disposed on the insulator.

Yet another aspect of the invention provides a leadless biostimulator, comprising a housing adapted to be implanted in or on a human heart, a first electrode and a second electrode coupled to the housing, a pulse generator disposed in the housing and electrically coupled to the first and second electrodes, the pulse generator configured to generate and deliver electrical pulses to heart tissue via the first and second electrodes, and a battery disposed in the housing and coupled to the pulse generator, the battery configured to supply energy for electrical pulse generation, wherein a loop area defined by a lead path from the first electrode to the second electrode and returning to the first electrode through the pulse generator is less than 1 cm².

In some embodiments, the loop area can be less than 0.7 cm².

In additional embodiments, a path length between the first and second electrodes is less than 10 cm. The path length can also be less than 2 cm.

In another aspect of the invention, the housing can have a total volume less than 1.5 cm³. In some embodiments, the housing can have a total volume less than 1.1 cm³.

The first electrode can be disposed on a flexible member. In some embodiments, the flexible member can comprise a fixation helix. In other embodiments, the fixation helix can be at least partially coated with an insulator, wherein the first electrode can comprise an uncoated portion of the fixation helix.

Another aspect of the invention provides an insulator disposed between the first and second electrodes. The insulator can be a coated portion of the housing. In some embodiments, the first electrode can be disposed on the insulator.

Yet another aspect of the invention provides for a method of operating a battery powered leadless biostimulator in or on the heart of the patient, comprising, performing an MRI procedure on the patient, and inducing a voltage in the leadless biostimulator less than 1.5 mV in response to the MRI procedure.

In some embodiments, the induced voltage is less than 0.25 mV.

In other embodiments, the MRI procedure does not generate heating of the leadless biostimulator sufficient to cause necrosis of heart tissue. For example, in some embodiments a temperature rise of less than 3 deg. C. is induced in the biostimulator in response to the MRI procedure.

In one embodiment, the step of performing a MRI procedure on the patient includes generating a pulsed gradient field with a magnetic field strength gradient of up to 50 mT/m. The pulsed gradient field can have a slew-rate of up to 20 T/sec.

In some embodiments, the biostimulator does not revert to asynchronous pacing during the MRI procedure.

Another aspect of the invention provides a method of obtaining an MRI image of a patient, the patient having an implanted battery powered leadless biostimulator, the method comprising generating a static magnetic field, a pulsed gradient field, and an RF field in the patient, maintaining safe operation of the leadless biostimulator within the patient in the presence of the static magnetic field, the gradient field, and the RF field without attenuating or eliminating a signal in the leadless biostimulator.

Yet another aspect of the invention provides a leadless biostimulator, comprising a housing adapted to be implanted in or on a human heart, a first electrode and a second electrode coupled to the housing, a pulse generator disposed in the housing and electrically coupled to the first and second electrodes, the pulse generator configured to generate and deliver electrical pulses to heart tissue via the first and second electrodes, and a battery disposed in the housing and coupled to the pulse generator, the battery configured to supply energy for electrical pulse generation; wherein the leadless biostimulator is configured for safe operation in or on the human heart during an MRI procedure without including an attenuation device to reduce or eliminate a signal in the leadless biostimulator during the MRI procedure.

In some embodiments, the attenuation device can be an RF filter, a fiber optic cable, an isolation system, or a band-stop filter. In other embodiments, the leadless biostimulator does not include a reed-switch.

Another aspect of the invention provides a method of performing an electrophysiological procedure on a heart, comprising operating a leadless biostimulator implanted in the heart and generating an induced voltage in the biostimulator of less than 1.5 mV during an MRI procedure without use of an attenuation device.

### BRIEF DESCRIPTION OF THE DRAWINGS

FIG. 1 is an implantable battery powered leadless biostimulator, according to one embodiment.

FIG. 2 is a top down view of an implantable battery powered leadless biostimulator, according to another embodiment.

FIG. 3 is a schematic drawing of the electronic components contained within an electronic compartment of a biostimulator, according to one embodiment.

FIGS. 4A and 4B are schematic drawings showing a loop area defined by a current path in a biostimulator, according to one embodiment.

FIG. 5 is a system including at least one biostimulator implanted on a heart and in communication with another device, according to one embodiment.

### DETAILED DESCRIPTION OF THE INVENTION

In some embodiments of a leadless biostimulator, a leadless cardiac pacemaker can communicate by conducted communication, representing a substantial departure from the conventional pacing systems. For example, an illustrative cardiac pacing system can perform cardiac pacing that has many of the advantages of conventional cardiac pacemakers while extending performance, functionality, and operating characteristics with one or more of several improvements.

In a particular embodiment of a cardiac pacing system, cardiac pacing is provided without a pulse generator located in the pectoral region or abdomen, without an electrode-lead separate from the pulse generator, without a communication coil or antenna, and without an additional requirement on battery power for transmitted communication.

Various embodiments of a system comprising one or more leadless cardiac pacemakers or biostimulators are described. An embodiment of a cardiac pacing system configured to attain these characteristics comprises a leadless cardiac pacemaker that is substantially enclosed in a hermetic housing suitable for placement on or attachment to the inside or outside of a cardiac chamber. The pacemaker can have at least two electrodes located within, on, or near the housing, for delivering pacing pulses to muscle of the cardiac chamber and optionally for sensing electrical activity from the muscle, and for bidirectional communication with at least one other device within or outside the body. The housing can contain a primary battery to provide power for pacing, sensing, and communication, for example bidirectional communication. The housing can optionally contain circuits for sensing cardiac activity from the electrodes. The housing contains circuits for receiving information from at least one other device via the electrodes and contains circuits for generating pacing pulses for delivery via the electrodes. The housing can optionally contain circuits for transmitting information to at least one other device via the electrodes and can optionally contain circuits for monitoring device health. The housing contains circuits for controlling these operations in a predetermined manner.

In accordance with some embodiments, a cardiac pacemaker can be adapted for implantation in the human body. In a particular embodiment, a leadless cardiac pacemaker can be adapted for implantation adjacent to the inside or outside wall of a cardiac chamber, using two or more electrodes located within, on, or within two centimeters of the housing of the pacemaker, for pacing the cardiac chamber upon receiving a triggering signal from at least one other device within the body.

For example, some embodiments of a leadless pacemaker can be configured for implantation adjacent to the inside or outside wall of a cardiac chamber without the need for a connection between the pulse generator and an electrode-lead, and without the need for a lead body.

Other example embodiments provide communication between the implanted leadless pulse generator and a device internal or external to the body, using conducted communication via the same electrodes used for pacing, without the need for an antenna or telemetry coil.

Some example embodiments can provide communication between the implanted leadless pacemaker pulse generator and a device internal or external to the body, with power requirements similar to those for cardiac pacing, to enable optimization of battery performance. In an illustrative embodiment, outgoing telemetry can be adapted to use no additional energy other than the energy contained in the pacing pulse. The telemetry function can be supplied via conducted communication using pacing and sensing electrodes as the operative structures for transmission and reception.

Self-contained or leadless pacemakers or other biostimulators are typically fixed to an intracardial implant site by an actively engaging mechanism such as a screw or helical member that screws into the myocardium. Examples of such leadless biostimulators are described in the following publications, the disclosures of which are incorporated by reference: (1) U.S. application Ser. No. 11/549,599, filed on Oct. 13, 2006, now U.S. Pat. No. 8,457,742, entitled “Leadless Cardiac Pacemaker System for Usage in Combination with an Implantable Cardioverter-Defibrillator”; (2) U.S. application Ser. No. 11/549,581 filed on Oct. 13, 2006, entitled “Leadless Cardiac Pacemaker”, and published as US2007/0088396A1 on Apr. 19, 2007; (3) U.S. application Ser. No. 11/549,591, filed on Oct. 13, 2006, entitled “Leadless Cardiac Pacemaker System with Conductive Communication” and published as US2007/0088397A1 on Apr. 19, 2007; (4) U.S. application Ser. No. 11/549,596 filed on Oct. 13, 2006, now U.S. Pat. No. 8,352,025, entitled “Leadless Cardiac Pacemaker Triggered by Conductive Communication”; (5) U.S. application Ser. No. 11/549,603 filed on Oct. 13, 2006, now U.S. Pat. No. 7,937,148, entitled “Rate Responsive Leadless Cardiac Pacemaker”; (6) U.S. application Ser. No. 11/549,605 filed on Oct. 13, 2006, now U.S. Pat. No. 7,945,333, entitled “Programmer for Biostimulator System”; (7) U.S. application Ser. No. 11/549,574, filed on Oct. 13, 2006, now U.S. Pat. No. 8,010,209, entitled “Delivery System for Implantable Biostimulator”; and (8) International Application No. PCT/US2006/040564, filed on Oct. 13, 2006, entitled “Leadless Cardiac Pacemaker and System” and published as WO07047681A2 on Apr. 26, 2007.

The biostimulators described herein are configured for safe operation under a wide range of MRI conditions. The biostimulators described herein have a total volume small enough to avoid excessive image artifacts during a MRI procedure. The biostimulators described herein have reduced path lengths between electrodes to minimize tissue heating at the site of the biostimulator. The biostimulators described herein also minimize the current loop area within the biostimulator to reduce an induced current and voltage in the biostimulator and prevent inappropriate sensing, triggering, and other problems associated with induced currents and voltages in biostimulators during MRI procedures.

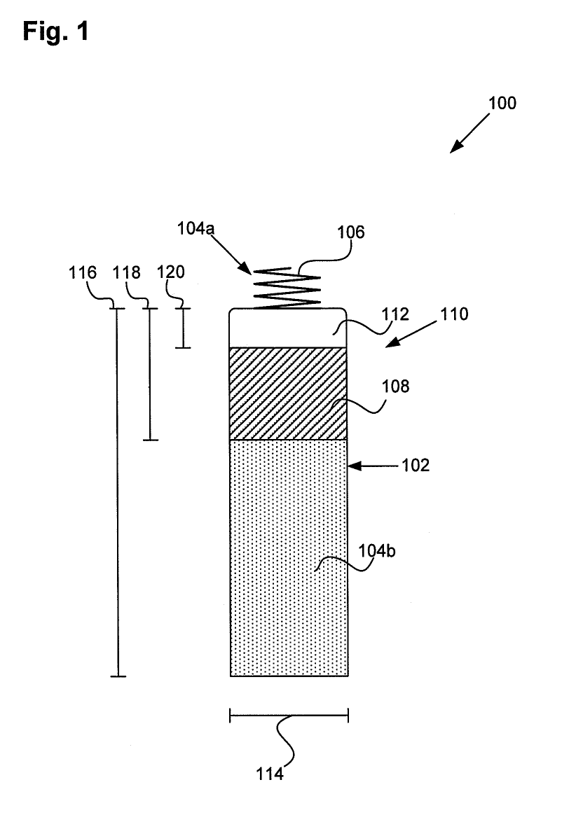

FIG. 1 shows a leadless cardiac pacemaker or leadless biostimulator 100 configured for safe operation during MRI over a wide range of MRI conditions. The biostimulators described herein and depicted variously in FIGS. 1-5 typically include a hermetic housing 102 with electrodes 104a and 104b disposed thereon, and an electronics compartment 110 within the housing containing the electronic components necessary for operation of the biostimulator. In one embodiment, the electronics compartment 110 can comprise approximately 25% of the internal volume of the hermetic housing, and a battery (not shown) can comprise approximately 75% of the internal volume of the housing. The hermetic housing can be adapted to be implanted on or in a human heart, and can be cylindrically shaped, rectangular, spherical, or any other appropriate shapes, for example.

The housing can comprise a conductive material such as titanium, 316L stainless steel, or other similar materials. In the case of 316L stainless steel, the housing can be annealed for the magnetic permeability to approach a value of 1. The housing can further comprise an insulator disposed on the conductive material to separate electrodes 104a and 104b. The insulator can be an insulative coating on a portion of the housing between the electrodes, and can comprise materials such as silicone, polyurethane, parylene, or another biocompatible electrical insulator commonly used for implantable medical devices. In some embodiments, a single insulator 108 is disposed along the portion of the housing between electrodes 104a and 104b. In some embodiments, the housing itself can comprise an insulator instead of a conductor, such as an alumina ceramic or other similar materials, and the electrodes can be disposed upon the housing.

As shown in FIG. 1, the biostimulator can further include a header assembly 112 to isolate electrode 104a from electrode 104b. The header assembly 112 can be made from Techothane or another biocompatible plastic, and can contain a ceramic to metal feedthrough, a glass to metal feedthrough, or other appropriate feedthrough insulator as known in the art.

The biostimulator 100 can include electrodes 104a and 104b. The electrodes can comprise pace/sense electrodes, reference, indifferent, or return electrodes. A low-polarization coating can be applied to the electrodes, such as platinum, platinum-iridium, iridium, iridium-oxide, titanium-nitride, carbon, or other materials commonly used to reduce polarization effects, for example.

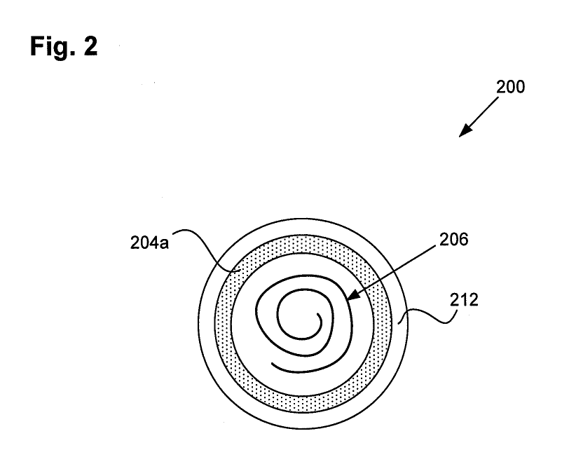

In FIG. 1, electrode 104a can be a pace/sense electrode and electrode 104b can be a reference, indifferent, or return electrode. As shown, electrode 104a can be disposed on a fixation device 106 and the electrode 104b can be disposed on the housing 102. The electrode 104b can be a portion of the conductive housing 102 that does not include an insulator 108. The fixation device can be a fixation helix or other flexible structure suitable for attaching the housing to tissue, such as heart tissue. In some embodiments, the electrode 104a can be disposed on the fixation device, such as a portion of the fixation device 106 that does not have an insulative coating. In other embodiments, the electrode 104a may be independent from the fixation device in various forms and sizes. For example, FIG. 2 shows a top down view of a biostimulator 200 having an annular or donut pace/sense electrode 204a disposed on a top portion of the header assembly 212. The biostimulator 200 can further include a second electrode (not shown) on the uncoated or uninsulated portion of the housing, similar to electrode 104b shown in FIG. 1. In the embodiment shown in FIG. 2, the fixation device is separate from the pace/sense electrode 204a.

Several techniques and structures can be used for attaching the housing 102 to the interior or exterior wall of the heart. A helical fixation device 106, as shown in FIG. 1, can enable insertion of the device endocardially or epicardially through a guiding catheter. A torqueable catheter can be used to rotate the housing and force fixation device into heart tissue, thus affixing the fixation device (and also the electrode 104a in FIG. 1) into contact with stimulable tissue. Electrode 104b can serve as an indifferent electrode for sensing and pacing. The fixation device may be coated for electrical insulation, and a steroid-eluting matrix may be included on or near the device to minimize fibrotic reaction, as is known in conventional pacing electrode-leads. In other configurations, suture holes (not shown) can be used to affix the housing directly to cardiac muscle with ligatures, during procedures where the exterior surface of the heart is exposed. Other attachment structures used with conventional cardiac electrode-leads including tines or barbs for grasping trabeculae in the interior of the ventricle, atrium, or coronary sinus may also be used in conjunction with or instead of the illustrative attachment structures.

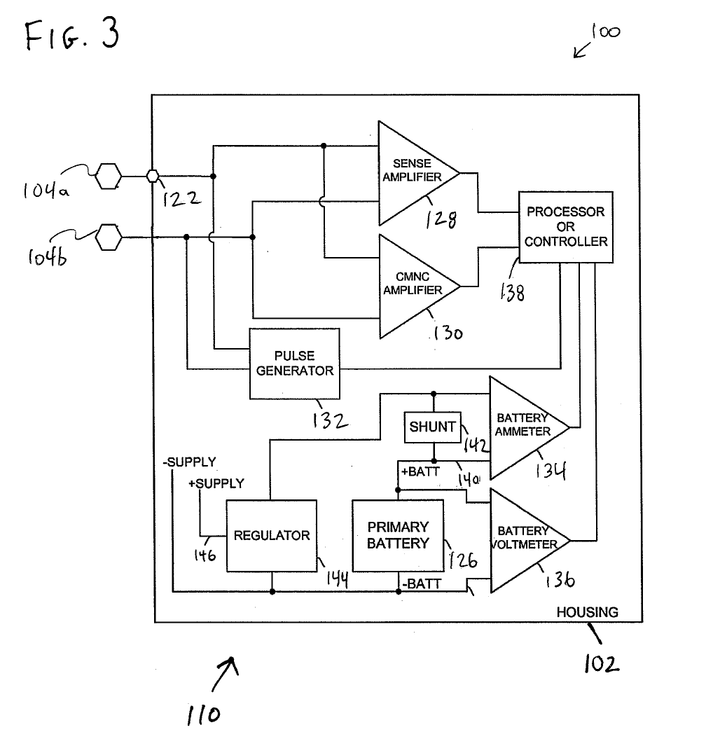

FIG. 3 is a schematic drawing of the electronic components that can be contained in electronic compartment of a biostimulator described herein. It should be understood that some components described below may not be required or included in all embodiments of the invention. As shown in FIG. 3, electronics compartment 110 of biostimulator 100 can be contained within a hermetic housing 102 configured for placement on or attachment to the inside or outside of a human heart. The electronics compartment can be coupled to at least two leadless electrodes 104a and 104b within, on, or proximal to the housing for delivering pacing pulses to and sensing electrical activity from the muscle of the cardiac chamber, and for bidirectional communication with at least one other device within or outside the body. A hermetic feedthrough 122 can conduct electrode signals through the housing 102. The housing can contain a primary battery 126 to supply power for pacing, sensing, and communication. The housing can also contain circuits 128 for sensing cardiac activity from the electrodes, circuits 130 for receiving information from at least one other device via the electrodes, and a pulse generator 132 configured to generate and deliver electrical pulses to heart tissue via the electrodes and also for transmitting information to at least one other device via the electrodes. The housing can further contain circuits for monitoring device health, for example a battery current monitor 134 and a battery voltage monitor 136, and can contain a controller 138 for controlling operations in a predetermined manner. Current from the positive terminal 140 of the primary battery can flow through a shunt 142 to a regulator circuit 144 to create a positive voltage supply 146 suitable for powering the remaining circuitry of the biostimulator 100. The shunt can enable the battery current monitor to provide the controller with an indication of battery current drain and indirectly of device health.

The total volume of the biostimulator 100 is typically less than 1.5 cm³, and preferably less than 1.2 cm³ to avoid excessive image artifacts within a patient during MRI. The total volume of the electronics compartment 110 is typically less than 0.4 cm³. Referring back to FIGS. 1-2, in a preferred embodiment, a cylindrical housing can have a diameter 114 of 0.7 cm and a length 116 of 2.8 cm for a total volume of approximately 1.1 cm³. In other embodiments, the diameter of the housing (or width/thickness of the housing if the housing is rectangular) can be approximately 0.4 to 1.0 cm and the length of the housing can be approximately 0.75 to 3.0 cm, resulting in a total volume ranging from 0.25 to 2.5 cm³. When the biostimulator includes an electrode disposed on the fixation device 106, the electrode can typically have an exposed surface area between 1 mm² and 8 mm².

The path length 118 between the electrodes 104a and 104b can affect the amount of RF field energy picked up by the biostimulator, which can result in tissue heating at the site of the electrode of the implanted biostimulator. In a preferred embodiment, the path length 118 between the electrodes is less than 2 cm and is preferably 1 cm. However, in other embodiments, the path length can be approximately 0.2 to 3.0 cm. It has been shown that a path length less than 10 cm between electrodes results in an acceptable temperature rise at the electrode tissue junction due to the RF field of MRI. It is an object of the biostimulator described herein to limit a temperature rise at the site of the electrode and tissue to less than 3° C. for safe operation within a patient during a MRI procedure. Still referring to FIG. 1, the biostimulator can also include a feedthrough distance 120 which is the distance from the pace/sense electrode (e.g., electrode 104a) to the insulated portion 108 of housing 102.

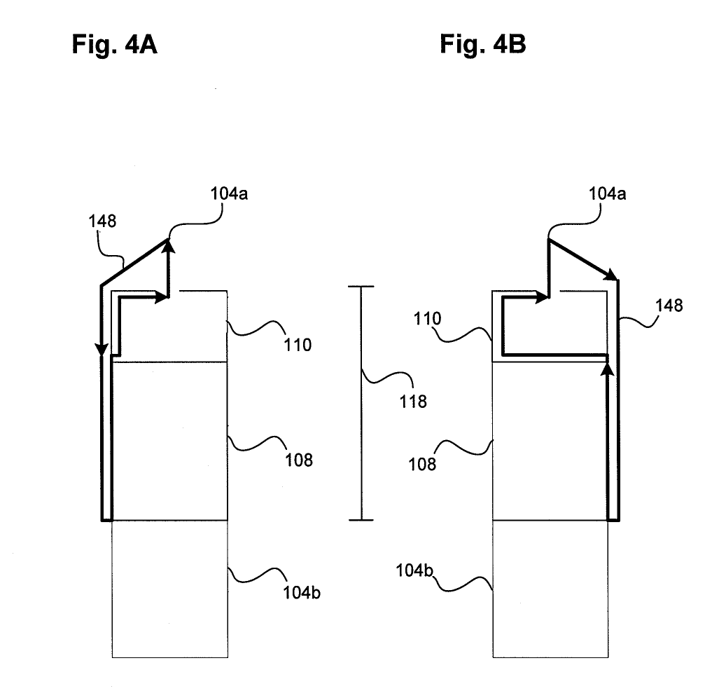

The loop area of the biostimulator 100 affects the amount of induced currents in the biostimulator. Referring now to FIGS. 4A and 4B, the path length 118 between electrodes 104a and 104b and the volume of the electronics compartment define a current loop area 148 in the biostimulator. FIG. 4A illustrates a minimum loop area 148, showing the lead path of the biostimulator starting at electrode 104a, flowing to electrode 104b, and returning to electrode 104a through the electronics compartment 110. FIG. 4B illustrates a maximum loop area 148 following a similar current path but taking the longest route through the electronics compartment. It can be seen then that the worst case loop area for magnetic induction in the biostimulator is the area of the electronics compartment. Thus, this loop area can be further minimized by minimizing the portion of the loop area within the electronics compartment. In a preferred embodiment of the invention, a biostimulator having a path length of 2 cm and an electronics compartment with a volume of 0.4 cm³ can result in a loop area of less than 1 cm² and preferably less than 0.7 cm². Compared to a conventional pacemaker system with a typical loop area of 200 cm², the biostimulator of the present invention can effectively reduce an induced voltage in the biostimulator by a factor of 275:1. This reduction can be significantly higher by carefully optimizing the layout of electronic components in the electronic compartment to minimize the effective loop area. In one embodiment, a voltage of less than 1.5 mV is induced in the biostimulator during an MRI procedure, and preferably a voltage of less than 0.25 mV is induced in the biostimulator during the MRI procedure.

Thus, the biostimulator of the present invention is configured for safe operation in or on the human heart during an MRI procedure by having a total volume small enough to avoid excessive image artifacts, by reducing the path length between electrodes to minimize tissue heating at the site of the electrode of the implanted biostimulator, and by minimizing the loop area of the biostimulator to minimize an induced current and voltage in the biostimulator to prevent inappropriate sensing, triggering, and other problems associated with induced currents and voltages in biostimulators during MRI procedures. The biostimulator described herein provides for safe operation under a wide range of MRI conditions without including an attenuation device or a “trap” circuit to reduce or eliminate signals in the biostimulator at one or more predetermined frequencies during the MRI procedure. These predetermined frequencies may be calculated from the Larmor frequency for protons (hydrogen nuclei) which is 42.58 MHz/T. For example, for a 3.0 T field, the predetermined frequency is 128 MHz. Attenuation devices used by other devices in an attempt to provide safe operation under MRI include an RF filter or shield, a fiber optic cable, an isolation system in conjunction with magnetic and RF sensors, or a band-stop filter, for example. Additionally, the leadless biostimulator described herein can be safely operated without requiring or including a reed-switch.

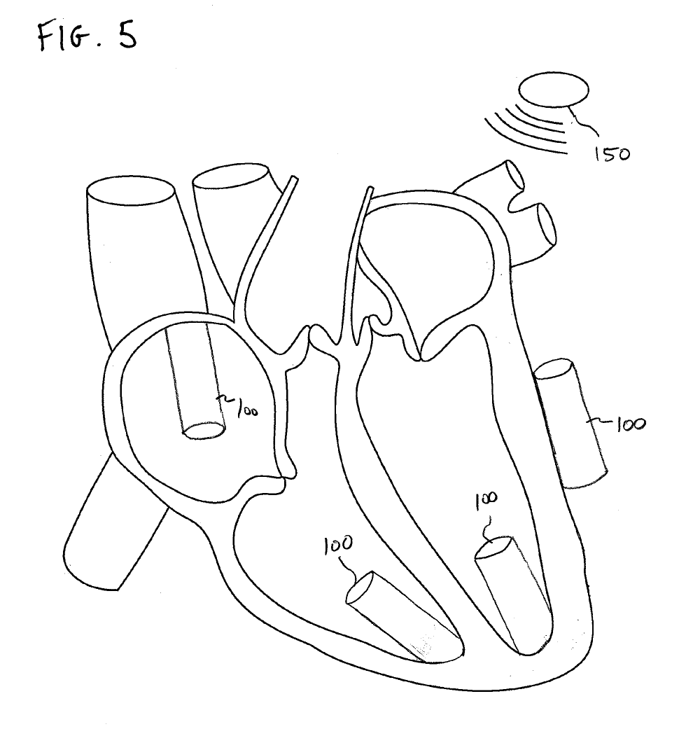

Referring to FIG. 5, a pictorial diagram shows one or more leadless cardiac biostimulators 100 with conducted communication for performing cardiac pacing in conjunction with another implantable device 150, such as an implantable cardioverter-defibrillator (ICD). The system can implement for example single-chamber pacing, dual-chamber pacing, or three-chamber pacing for cardiac resynchronization therapy, without requiring pacing lead connections to the defibrillator. Although FIG. 5 shows leadless cardiac biostimulators placed in multiple heart chambers as well as placed epicardially along the muscle, in other embodiments the biostimulators can be used in only a single chamber or, alternatively, be placed only on the epicardium. Furthermore, in other embodiments, the biostimulators can be used without an ICD.

The leadless cardiac pacemakers 100 can communicate with one another and/or communicate with a non-implanted programmer and/or the implanted ICD 150 via the same electrodes that are also used to deliver pacing pulses. Usage of the electrodes for communication enables the one or more leadless cardiac pacemakers for antenna-less and telemetry coil-less communication.

Methods of operating a leadless pacemaker or biostimulator under a wide range of MRI conditions will now be discussed.

In one method of the invention, a battery powered leadless biostimulator is operated in or on the heart of the patient. The biostimulator can comprise any biostimulator described herein. While the biostimulator is operating in the patient, an MRI procedure can be performed on the patient. As a result of the MRI procedure, a voltage of less than 1.5 mV and preferably less than 0.25 mV is induced in the leadless biostimulator in response to the MRI procedure. In some embodiments, the voltage induced in the biostimulator is reduced by minimizing a loop area in the biostimulator. In other embodiments, the voltage induced is reduced by minimizing a path length between electrodes disposed on the biostimulator. In yet other embodiments, the voltage induced is reduced by minimizing both the loop area and the path length in the biostimulator.

In another embodiment of the invention, operating the biostimulator in a patient during a MRI procedure does not generate heating of an electrode on the biostimulator sufficient to cause necrosis of heart tissue. The temperature rise in the biostimulator as a result of the MRI procedure can be less than 3° C., for example.

In another embodiment of the method, the biostimulator does not revert to asynchronous pacing during the MRI procedure.

The step of performing a MRI procedure may include generating a pulsed gradient field with a magnetic field strength gradient of up to 50 mT/m, wherein the pulsed gradient field has a slew-rate of up to 20 T/sec, for example.

Another method of the invention comprises a method of obtaining an MRI image of a patient having an implanted battery powered leadless biostimulator. The method can include the step of generating a static magnetic field, a pulsed gradient field, and an RF field in the patient, and maintaining safe operation of the leadless biostimulator within the patient in the presence of the static magnetic field, the gradient field, and the RF field without attenuating or eliminating a signal in the leadless biostimulator. In some embodiments of the method, a voltage induced in the biostimulator is less than 1.5 mV and preferably less than 0.25 mV, for example,

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A leadless biostimulator, comprising:
a housing adapted to be implanted in or on a human heart;
a first electrode and a second electrode coupled to the housing;
a pulse generator disposed in the housing and electrically coupled to the first and second electrodes, the pulse generator configured to generate and deliver electrical pulses to heart tissue via the first and second electrodes; and
a battery disposed in the housing and coupled to the pulse generator, the battery configured to supply energy for electrical pulse generation;
wherein a loop area defined by a lead path from the first electrode to the second electrode and returning to the first electrode through the pulse generator is less than 1 cm².

2. The leadless biostimulator of claim 1 wherein the loop area is less than 0.7 cm².

3. The leadless biostimulator of claim 1 wherein a path length between the first and second electrodes is less than 10 cm.

4. The leadless biostimulator of claim 3 wherein the path length is less than 2 cm.

5. The leadless biostimulator of claim 1 wherein the housing has a total volume less than 1.5 cm³.

6. The leadless biostimulator of claim 1 wherein the housing has a total volume less than 1.1 cm³.

7. The leadless biostimulator of claim 1 wherein the first electrode comprises a pace/sense electrode.

8. The leadless biostimulator of claim 7 wherein the second electrode comprises a return electrode.

9. The leadless biostimulator of claim 1 wherein the first electrode comprises a fixation helix.

10. The leadless biostimulator of claim 1 wherein the first electrode comprises a can electrode.

11. The leadless biostimulator of claim 1 wherein the first electrode includes a low-polarization coating.

12. The leadless biostimulator of claim 1 wherein the second electrode includes a low-polarization coating.

13. The leadless biostimulator of claim 1 further comprising an insulator disposed between the first and second electrodes.

14. The leadless biostimulator of claim 13 wherein the first electrode is disposed on the insulator.
